(19) United States
(12) Patent Application Publication
(10) Pub. No.: US 2026/0054043 A1
(43) Pub. Date: Feb. 26, 2026

(54) METHOD AND APPARATUS FOR MEDICAL INJECTION

(71) Applicant: BOVONEX INC., Richmond (CA)

(72) Inventors: Ali POORKHALIL, Tehran (IR); Vahid SAHIHOLNASAB, Richmond (CA); Mehdi OMIDBAKHSH, Markham (CA); Hasan FARROKHZAD, Tehran (IR)

(73) Assignee: BOVONEX INC., Richmond, BC (CA)

(21) Appl. No.: 18/840,935

(22) PCT Filed: Aug. 13, 2023
(86) PCT No.: PCT/IB2023/058163

§ 371 Date: Aug. 23, 2024

(51) Int. Cl. A61M 37/00; A61M 39/00; A61M 39/22

(52) U.S. Cl. CPC A61M 37/0015; A61M 39/22; A61M 2037/0023; A61M 2037/003; A61M 2037/0061; A61M 2039/0027; A61M 2202/0007; A61M 2202/049; A61M 2205/0216; A61M 2210/04

(57) ABSTRACT

A medical injection apparatus includes a first reservoir, a second reservoir, a static mixer, and an injection unit. The first reservoir contains a saline solution. The second reservoir contains medicine. The static mixer receives the saline solution from the first reservoir, receives the medicine from the second reservoir, and obtains a medical solution by mixing the medicine with the saline solution. The injection unit receives the medical solution from the static mixer and injects the medical solution into a skin of a subject.

### TECHNICAL FIELD

The present disclosure generally relates to drug delivery, and particularly, to medical injection.

### BACKGROUND ART

Transdermal drug delivery (TDD) is a noninvasive transportation of drugs across patients' skins and may have several advantages over traditional methods, such as oral drug delivery or hypodermic injection. TDD may provide a simple yet reliable mechanism for administering drugs. In addition, TDD may be self-administered due to its noninvasive and painless nature.

Microneedles are micron-scaled needles that have been utilized for TDD [10,463,608 B2, 11,311,496 B2, 11,628,208 B2]. Microneedles may be fabricated in different sizes and types, thereby allowing for drug administration for different drug types, injection sites, and depths of skin where drugs are injected. However, efficient and safe usage of microneedles for drug delivery may require preparing an appropriate dosage of drugs that are to be delivered through microneedles, which may limit a wide-spread usage of these devices by non-expert users. Resolving such limitation may lead to significant reduction of treatment costs. There is, therefore, a need for a method and an apparatus that may provide an appropriate dosage of drugs for drug delivery utilizing microneedles.

### SUMMARY OF THE INVENTION

This summary is intended to provide an overview of the subject matter of this patent, and is not intended to identify essential elements or key elements of the subject matter, nor is it intended to be used to determine the scope of the claimed implementations. The proper scope of this patent may be ascertained from the claims set forth below in view of the detailed description below and the drawings.

In one general aspect, the present disclosure describes an exemplary medical injection apparatus. An exemplary medical injection apparatus may include a first reservoir, a second reservoir, a static mixer, and an injection unit. An exemplary first reservoir may contain a saline solution. An exemplary second reservoir may contain medicine. An exemplary static mixer may receive the saline solution from the first reservoir, receive the medicine from the second reservoir, and obtain a medical solution by mixing the medicine with the saline solution. An exemplary injection unit may receive the medical solution from the static mixer and inject the medical solution into a skin of a subject.

An exemplary first reservoir may be coupled in parallel with the second reservoir. An exemplary medical injection apparatus may further include a valve that may be connected between the first reservoir, the second reservoir, and the static mixer. An exemplary valve may seal each of the first reservoir, the second reservoir, and the static mixer with respect to one another responsive to the valve being closed. An exemplary valve may pass the saline solution from the first reservoir and may pass the medicine from the second reservoir to the static mixer responsive to the valve being opened. An exemplary medicine may include a liquid medicine. An exemplary liquid medicine may include a solution of botulinum toxin.

In an exemplary embodiment, the first reservoir may be mounted on the second reservoir and the second reservoir may be mounted on the static mixer. In an exemplary embodiment, the first reservoir may include an elastic balloon. An exemplary medical injection apparatus may further include one of a barrier or a semipermeable membrane that may be placed between the elastic balloon and the second reservoir. In an exemplary embodiment, each of the barrier and the semipermeable membrane may pass the saline solution from the elastic balloon to the second reservoir responsive to pressing the elastic balloon.

An exemplary medical injection apparatus may further include a valve that may be connected between the first reservoir and the second reservoir. An exemplary valve may seal the first reservoir with respect to the second reservoir responsive to the valve being closed and may push the medicine from the second reservoir to the static mixer by passing the saline solution from the first reservoir to the second reservoir responsive to the valve being opened. An exemplary medicine may include a powder. An exemplary powder may include a botulinum toxin powder.

An exemplary injection unit may include one or more chambers, a primary pipe, one or more secondary pipes, and a plurality of microneedle arrays. An exemplary primary pipe may be connected to the static mixer. In an exemplary embodiment, the one or more secondary pipes may connect the primary pipe to the one or more chambers. In an exemplary embodiment, each respective secondary pipe of the one or more secondary pipes may receive the medical solution from the static mixer through the primary pipe and may pass the medical solution to a respective chamber of the one or more chambers. In an exemplary embodiment, the plurality of microneedle arrays may be attached to the one or more chambers. An exemplary tip of each respective microneedle in each respective microneedle array of the plurality of microneedle arrays may extrude from a surface of each respective chamber of the one or more chambers.

An exemplary medical injection apparatus may further include a valve and a compressed spring. An exemplary valve may be connected between the static mixer and the primary pipe. An exemplary compressed spring may be mounted on the first reservoir. In an exemplary embodiment, the compressed spring may push the medical solution from the static mixer into the primary pipe responsive to the valve being opened.

An exemplary medical injection apparatus may further include one or more elastic membranes. In an exemplary embodiment, each respective elastic membrane of the one or more elastic membranes may be mounted on a respective chamber of the one or more chambers and may push the medical solution from the respective chamber into each microneedle in a respective microneedle array of the plurality of microneedle arrays responsive to the respective elastic membrane being pushed.

An exemplary medical injection apparatus may further include a plurality of cartridges. In an exemplary embodiment, each respective cartridge of the plurality of cartridges may be connected between a respective chamber of the one or more chambers and a respective microneedle in a respective microneedle array of the plurality of microneedle arrays. In an exemplary embodiment, each respective cartridge of the plurality of cartridges may receive a predetermined amount of the medical solution from the respective chamber and pass the predetermined amount of the medical solution to the respective microneedle.

Other exemplary systems, methods, features and advantages of the implementations will be, or will become, apparent to one of ordinary skill in the art upon examination of the following figures and detailed description. It is intended that all such additional systems, methods, features and advantages be included within this description and this summary, be within the scope of the implementations, and be protected by the claims herein.

### BRIEF DESCRIPTION OF DRAWINGS

The drawing figures depict one or more implementations in accord with the present teachings, by way of example only, not by way of limitation. In the figures, like reference numerals refer to the same or similar elements.

FIG. 1 shows a schematic of a medical injection apparatus, consistent with one or more exemplary embodiments of the present disclosure.

FIG. 2 shows a schematic of a medical injection apparatus with parallel reservoirs, consistent with one or more exemplary embodiments of the present disclosure.

FIG. 3 shows a schematic of a medical injection apparatus with reservoirs connected in series, consistent with one or more exemplary embodiments of the present disclosure.

FIG. 4 shows a schematic of a medical injection apparatus with an elastic balloon mounted on a reservoir, consistent with one or more exemplary embodiments of the present disclosure.

FIG. 5 shows a schematic of an injection unit, consistent with one or more exemplary embodiments of the present disclosure.

FIG. 6A shows a schematic of a chamber reservoir, consistent with one or more exemplary embodiments of the present disclosure.

FIG. 6B shows a schematic of a chamber interface, consistent with one or more exemplary embodiments of the present disclosure.

FIG. 7 shows a schematic of different hollow microneedles, consistent with one or more exemplary embodiments of the present disclosure.

FIG. 8 shows a schematic of an injection unit connected to a static mixer through a valve, consistent with one or more exemplary embodiments of the present disclosure.

FIG. 9 shows a schematic of a medical injection apparatus with a compressed spring mounted on reservoirs, consistent with one or more exemplary embodiments of the present disclosure.

FIG. 10 shows a schematic of an elastic membrane mounted on a chamber, consistent with one or more exemplary embodiments of the present disclosure.

FIG. 11 shows a schematic of a cartridge connected between a chamber and a microneedle, consistent with one or more exemplary embodiments of the present disclosure.

### DETAILED DESCRIPTION OF THE INVENTION

In the following detailed description, numerous specific details are set forth by way of examples in order to provide a thorough understanding of the relevant teachings. However, it should be apparent that the present teachings may be practiced without such details. In other instances, well known methods, procedures, components, and/or circuitry have been described at a relatively high-level, without detail, in order to avoid unnecessarily obscuring aspects of the present teachings.

The following detailed description is presented to enable a person skilled in the art to make and use the methods and devices disclosed in exemplary embodiments of the present disclosure. For purposes of explanation, specific nomenclature is set forth to provide a thorough understanding of the present disclosure. However, it will be apparent to one skilled in the art that these specific details are not required to practice the disclosed exemplary embodiments. Descriptions of specific exemplary embodiments are provided only as representative examples. Various modifications to the exemplary implementations will be readily apparent to one skilled in the art, and the general principles defined herein may be applied to other implementations and applications without departing from the scope of the present disclosure. The present disclosure is not intended to be limited to the implementations shown, but is to be accorded the widest possible scope consistent with the principles and features disclosed herein.

Herein is disclosed an exemplary apparatus for medical injection. An exemplary apparatus may include a pair of reservoirs for containing saline solution and medicine, a static mixer for mixing the saline solution with the medicine for providing an appropriate dosage of drug (i.e., a medical solution), and an injection unit that may inject the provided drug into a patient's skin. Two exemplary reservoirs may be connected in parallel or in series with respect to the static mixer. Exemplary contents of the reservoirs may pass to the static mixer either by opening a valve that may be placed between the pair of reservoirs and the static mixer or by flowing the saline solution into a reservoir that contains the medicine, thereby pushing the medicine along with the saline solution into the static mixer. An exemplary injection unit may include one or more chambers which may receive and store the prepared drug. Exemplary microneedles may have been attached to the chambers to inject a desired amount of drug into a patient's skin on demand. As a result, an exemplary apparatus may provide an appropriate dosage of drugs to be injected. Therefore, since an exemplary apparatus may be manufactured as a stand-alone device, it may facilitate self-administration of drugs by non-expert users. An exemplary drug may include several types of toxin that may be injected into the skin, such as botulinum toxin.

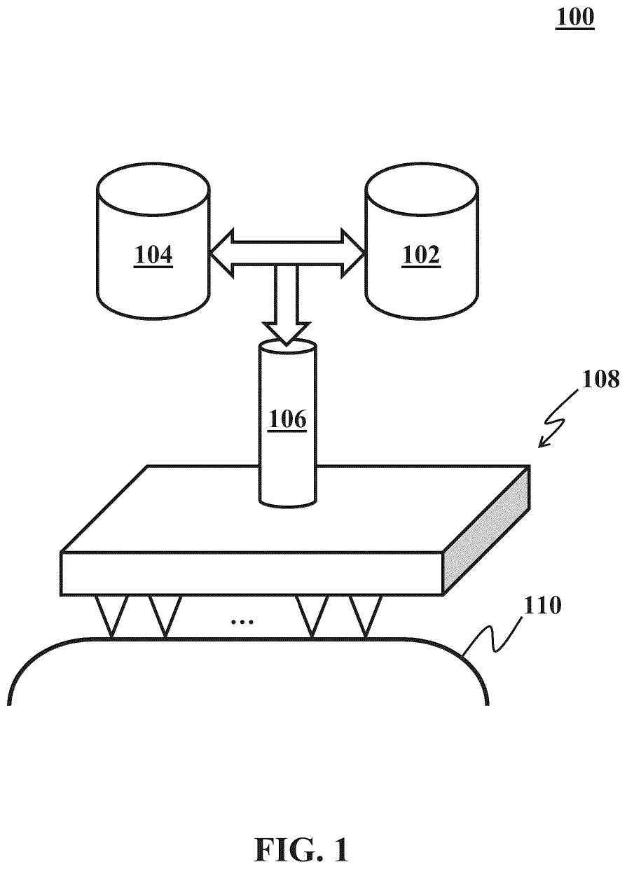

FIG. 1 shows a schematic of a medical injection apparatus, consistent with one or more exemplary embodiments of the present disclosure. An exemplary medical injection apparatus 100 may include a first reservoir 102, a second reservoir 104, a static mixer 106, and an injection unit 108. In an exemplary embodiment, first reservoir 102 may contain a saline solution. In an exemplary embodiment, second reservoir 104 may contain medicine. In an exemplary embodiment, static mixer 106 may receive the saline solution from first reservoir 102 and may receive the medicine from second reservoir 104. In an exemplary embodiment, static mixer 106 may refer to a precision engineered device without moving components that may obtain a medical solution by mixing the medicine with the saline solution. An exemplary energy that may be required for mixing the medicine with the saline solution may come from a loss in pressure as the saline solution flows through static mixer 106. In an exemplary embodiment, injection unit 108 may receive the medical solution from static mixer 106. In an exemplary embodiment, injection unit 108 may inject the medical solution into a skin 110 of a subject.

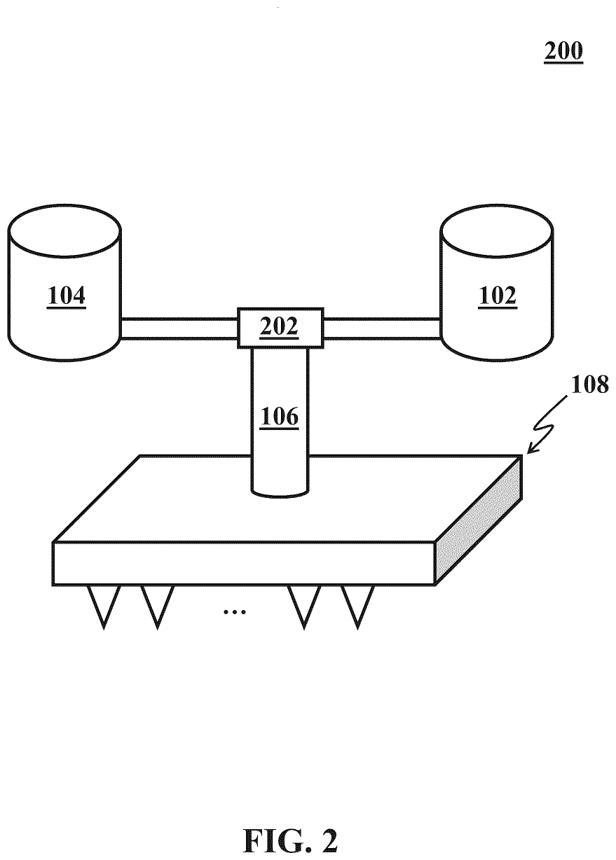

FIG. 2 shows a schematic of a medical injection apparatus with parallel reservoirs, consistent with one or more exemplary embodiments of the present disclosure. An exemplary medical injection apparatus 200 may be an implementation of medical injection apparatus 100. In an exemplary embodiment, first reservoir 102 may be coupled in parallel with second reservoir 104. In an exemplary embodiment, medical injection apparatus 200 may further include a valve 202. In an exemplary embodiment, valve 202 may be connected between first reservoir 102, second reservoir 104, and static mixer 106. In an exemplary embodiment, valve 202 may seal each of first reservoir 102, second reservoir 104, and static mixer 106 with respect to one another responsive to valve 202 being closed. Therefore, exemplary contents of first reservoir 102 may be kept separated from contents of second reservoir 104 when valve 202 is closed. In an exemplary embodiment, valve 202 may pass the saline solution from first reservoir 102 and may pass the medicine from second reservoir 104 to static mixer 106 responsive to the valve being opened. As a result, an exemplary saline solution in first reservoir 102 and an exemplary medicine in second reservoir 104 may pass into static mixer 106 when valve 202 is opened. An exemplary medicine may include a liquid medicine. An exemplary liquid medicine may include a solution of botulinum toxin.

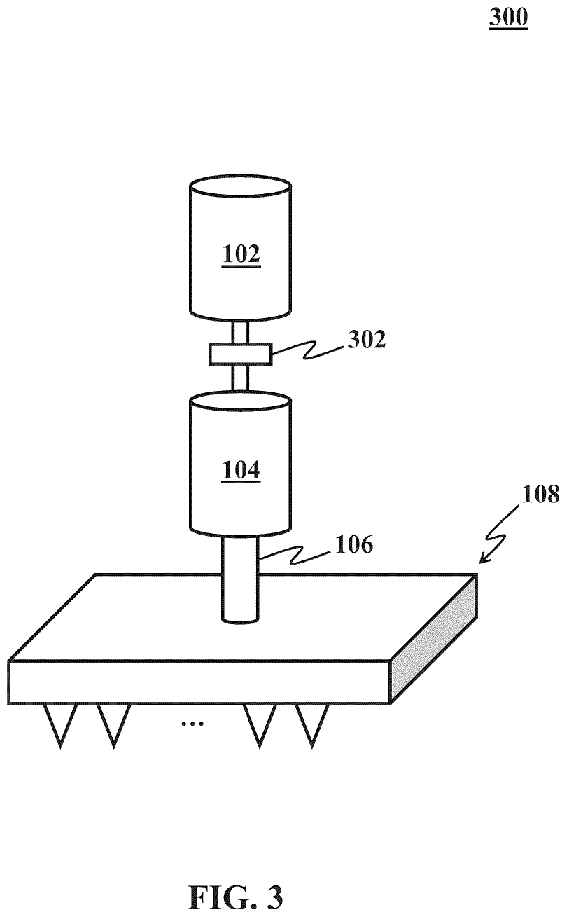

FIG. 3 shows a schematic of a medical injection apparatus with reservoirs connected in series, consistent with one or more exemplary embodiments of the present disclosure. An exemplary medical injection apparatus 300 may be an implementation of medical injection apparatus 100. In an exemplary embodiment, first reservoir 102 may be mounted on second reservoir 104 and second reservoir 104 may be mounted on static mixer 106. In an exemplary embodiment, medical injection apparatus 300 may further include a valve 302. In an exemplary embodiment, valve 302 may be connected between first reservoir 102 and second reservoir 104. In an exemplary embodiment, the medicine in second reservoir 104 may include a powder. An exemplary powder may include a botulinum toxin powder. In an exemplary embodiment, valve 302 may seal first reservoir 102 with respect to second reservoir 104 responsive to valve 302 being closed. Therefore, exemplary contents of first reservoir 102 may be kept separated from contents of second reservoir 104 when valve 302 is closed. In an exemplary embodiment, valve 302 may push the medicine from second reservoir 104 to static mixer 106 by passing the saline solution from first reservoir 102 to second reservoir 104 responsive to valve 302 being opened. As a result, an exemplary saline solution in first reservoir 102 and an exemplary medicine (for example, botulinum toxin powder) in second reservoir 104 may pass into static mixer 106 when valve 202 is opened.

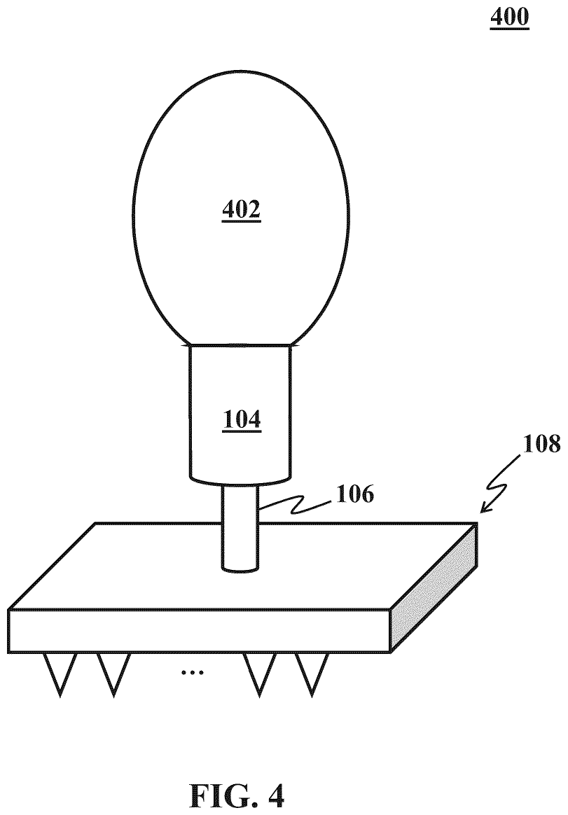

FIG. 4 shows a schematic of a medical injection apparatus with an elastic balloon mounted on a reservoir, consistent with one or more exemplary embodiments of the present disclosure. An exemplary medical injection apparatus 400 may be an implementation of medical injection apparatus 100. In an exemplary embodiment, first reservoir 102 may include an elastic balloon 402. In an exemplary embodiment, elastic balloon 402 may be mounted on second reservoir 104 and second reservoir 104 may be mounted on static mixer 106. An exemplary medicine may be pushed into static mixer 106 by pressing elastic balloon 402 so that the saline solution may flow from elastic balloon 402 into second reservoir 104, thereby pushing the medicine (for example, botulinum toxin powder) along with the saline solution into static mixer 106. In an exemplary embodiment, a pressure inside elastic balloon 402 may be varied so that the saline solution may flow from elastic balloon 402 into second reservoir 104, thereby pushing the medicine (for example, botulinum toxin powder) along with the saline solution into static mixer 106, as described below. In an exemplary embodiment, medical injection apparatus 400 may further include a barrier or a semipermeable membrane that may be placed between elastic balloon 402 and second reservoir 104. In an exemplary embodiment, each of the barrier and the semipermeable membrane may pass the saline solution from elastic balloon 402 to second reservoir 104 responsive to pressing elastic balloon 402. An exemplary barrier may be ruptured or burst when elastic balloon 402 is pressed, thereby passing the saline solution from elastic balloon 402 to second reservoir 104. An exemplary semipermeable membrane (such as a porous super hydrophobic membrane) may pass the saline solution from elastic balloon 402 to second reservoir 104 only when elastic balloon 402 is pressed.

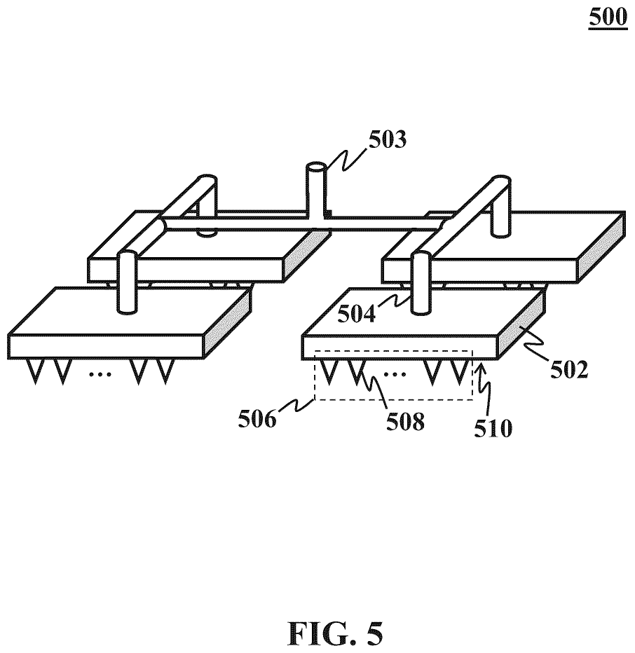

FIG. 5 shows a schematic of an injection unit, consistent with one or more exemplary embodiments of the present disclosure. An exemplary injection unit 500 may be an implementation of injection unit 108. In an exemplary embodiment, injection unit 500 may include one or more chambers (for example, a chamber 502), a primary pipe 503, one or more pipes (for example, a secondary pipe 504), and a plurality of microneedle arrays (for example, a microneedle array 506). In an exemplary embodiment, primary pipe 503 may be connected to static mixer 106 (not shown in FIG. 5). In an exemplary embodiment, the one or more secondary pipes may connect primary pipe 503 to the one or more chambers. In an exemplary embodiment, each respective secondary pipe of the one or more secondary pipes may receive the medical solution from static mixer 106 through primary pipe 503 and may pass the medical solution to a respective chamber of the one or more chambers. For example, secondary pipe 504 may receive the medical solution through primary pipe 503 and may pass it to chamber 502.

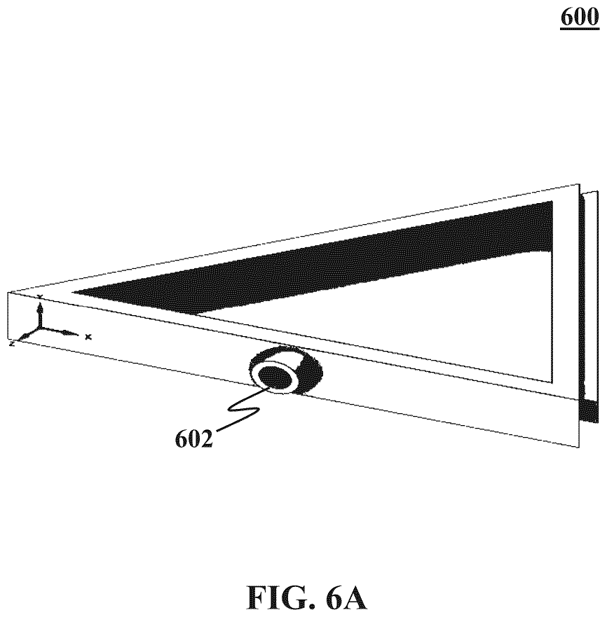

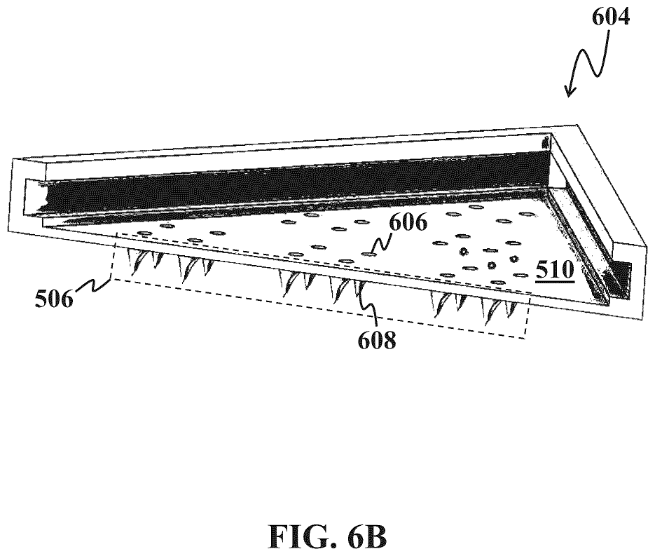

FIG. 6A shows a schematic of a chamber reservoir, consistent with one or more exemplary embodiments of the present disclosure. In an exemplary embodiment, chamber 502 may include different types and shapes. In an exemplary embodiment, chamber 502 may include a chamber reservoir 600 that may store the medical solution that is received from static mixer 106. An exemplary opening 602 may connect secondary pipe 504 to chamber reservoir 600 so that the medical solution may flow into chamber reservoir 600 from secondary pipe 504.

FIG. 6B shows a schematic of a chamber interface, consistent with one or more exemplary embodiments of the present disclosure. In an exemplary embodiment, chamber 502 may further include a chamber interface 604. Referring to FIGS. 6A and 6B, in an exemplary embodiment, chamber reservoir 600 may slide into chamber interface 604 so that contents of chamber reservoir 600 may be sealed from outside chamber 502.

Referring again to FIG. 5, in an exemplary embodiment, the plurality of microneedle arrays may be attached to the one or more chambers. For example, microneedle array 506 may be attached to chamber 502. An exemplary tip of each respective microneedle in each respective microneedle array of the plurality of microneedle arrays may extrude from a surface of each respective chamber of the one or more chambers. For example, a tip of an exemplary microneedle 508 in microneedle array 506 may extrude from a bottom surface 510 of chamber 502. Referring to FIGS. 5 and 6B, in an exemplary embodiment, a plurality of holes (for example, a hole 606) may implemented on bottom surface 510. An exemplary opening of each microneedle of microneedle array 506 may be attached to bottom surface 510 may be attached to bottom surface 510 below a corresponding hole of the plurality of holes. For example, a microneedle 608 may be attached below hole 604. As a result, an exemplary medical solution may flow into microneedle array 506 through the plurality of holes. In an exemplary embodiment, microneedle array 506 may include hollow microneedles. In an exemplary embodiment, a “hollow microneedle” may refer to a microneedle which may contain a reservoir that delivers the medical solution directly into a subject's body.

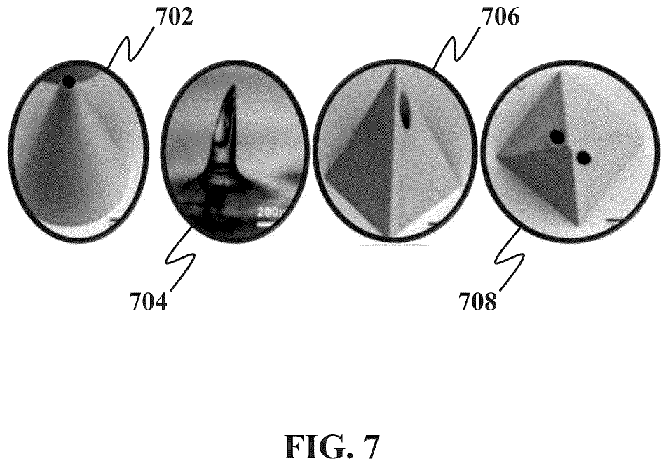

FIG. 7 shows a schematic of different hollow microneedles, consistent with one or more exemplary embodiments of the present disclosure. An exemplary microneedle 702 may have a conical shape and may have a bore in a middle of microneedle 702. An exemplary microneedle 704 may have a conical shape and may have a bore on a side of microneedle 704. An exemplary microneedle 706 may have a pyramid shape and may have a bore on a side of microneedle 706. An exemplary microneedle 708 may have a pyramid shape and may have more than one bore on different sides of microneedle 708.

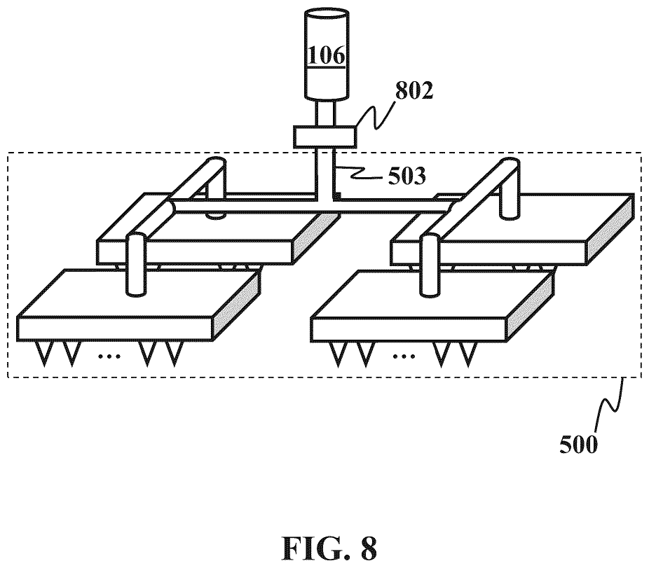

FIG. 8 shows a schematic of an injection unit connected to a static mixer through a valve, consistent with one or more exemplary embodiments of the present disclosure. In an exemplary embodiment, medical injection apparatus 100 (or implementations 200, 300, and 400) may further include a valve 802. In an exemplary embodiment, valve 802 may be connected between static mixer 106 and primary pipe 503. Referring to FIGS. 4 and 8, in an exemplary embodiment, opening valve 802 may cause elastic balloon 402 to push the saline solution into second reservoir 104 due to a higher pressure inside elastic balloon 402 than outside elastic balloon 402. Therefore, an exemplary medicine (for example, botulinum toxin powder) may be pushed into static mixer 106 along with the saline solution by opening valve 802. Consequently, an exemplary medical solution that is obtained in static mixer 106 may flow into primary pipe 503.

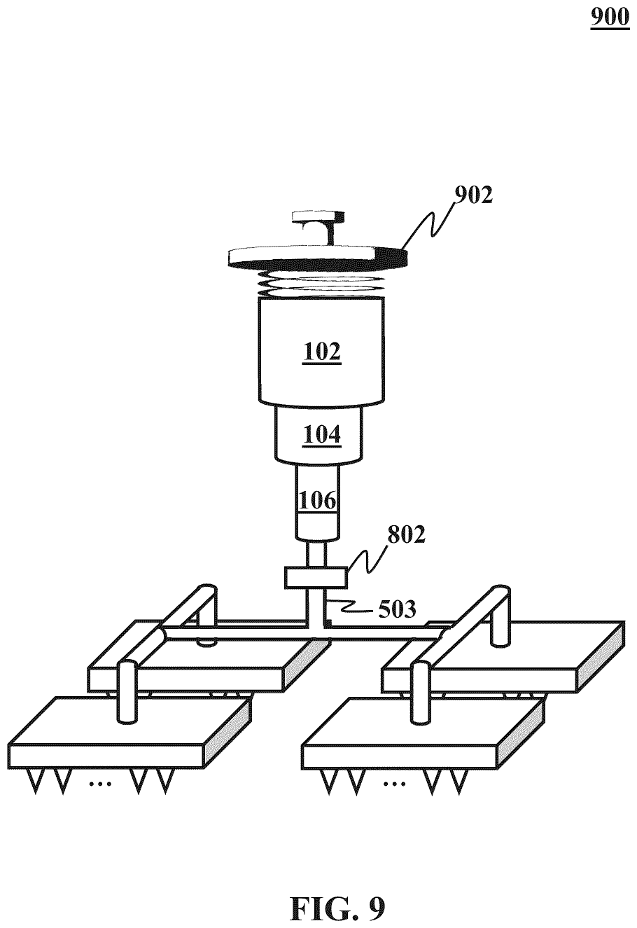

FIG. 9 shows a schematic of a medical injection apparatus with a compressed spring mounted on reservoirs, consistent with one or more exemplary embodiments of the present disclosure. An exemplary medical injection apparatus 900 may be an implementation of medical injection apparatus 100. In an exemplary embodiment, medical injection apparatus 900 may further include a compressed spring 902. In an exemplary embodiment, compressed spring 902 may be mounted on first reservoir 102. In an exemplary embodiment, compressed spring 902 may apply a force to the saline solution in first reservoir 102, which may consequently push the medicine in second reservoir 104 to valve 802. Therefore, in an exemplary embodiment, opening valve 802 may cause compressed spring 902 to expand by pushing the saline solution in first reservoir 102 into second reservoir 104 and consequently, pushing the saline solution and the medicine from second reservoir 104 into static mixer 106. Afterwards, an exemplary medical solution that is obtained in static mixer 106 may flow into primary pipe 503. Therefore, in an exemplary embodiment, compressed spring 902 may push the medical solution from static mixer 106 into primary pipe 503 responsive to valve 802 being opened.

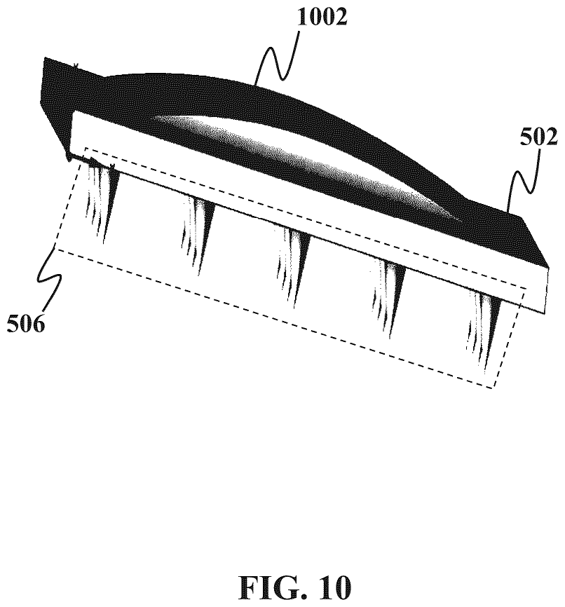

FIG. 10 shows a schematic of an elastic membrane mounted on a chamber, consistent with one or more exemplary embodiments of the present disclosure. In an exemplary embodiment, medical injection apparatus 100 may further include one or more elastic membranes (for example, elastic membrane 1002). Referring to FIGS. 1, 5, and 10, in an exemplary embodiment, each respective elastic membrane of the one or more elastic membranes may be mounted on a respective chamber of the one or more chambers. For example, elastic membrane 1002 may be mounted on chamber 502. In an exemplary embodiment, each respective elastic membrane of the one or more elastic membranes may push the medical solution from the respective chamber into a respective microneedle in a respective microneedle array of the plurality of microneedle arrays responsive to the respective elastic membrane being pushed. For example, elastic membrane 1002 may push the medical solution from chamber 502 into each microneedle in microneedle array 506 when elastic membrane 1002 is pushed.

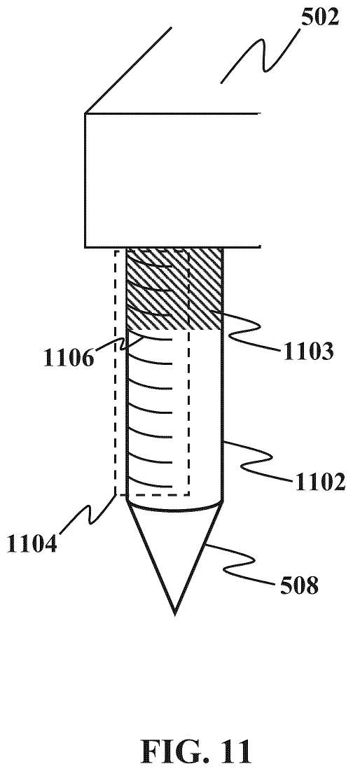

FIG. 11 shows a schematic of a cartridge connected between a chamber and a microneedle, consistent with one or more exemplary embodiments of the present disclosure. Referring to FIGS. 5 and 11, In an exemplary embodiment, medical injection apparatus 100 may further include a plurality of cartridges (for example, a cartridge 1102). In an exemplary embodiment, each respective cartridge of the plurality of cartridges may be connected between a respective chamber of the one or more chambers and a respective microneedle in a respective microneedle array of the plurality of microneedle arrays. For example, cartridge 1102 may be connected between chamber 502 and microneedle 508.

In an exemplary embodiment, each respective cartridge of the plurality of cartridges may receive a predetermined amount of the medical solution from the respective chamber and pass the predetermined amount of the medical solution to the respective microneedle. For example, cartridge 1102 may receive a predetermined amount 1103 of the medical solution from chamber 502. In an exemplary embodiment, predetermined amount 1103 may be determined by a medical expert prior to using medical injection apparatus 100. In an exemplary embodiment, cartridge 1102 may have graduated marks 1104 that indicate an amount of medical solution that may flow into cartridge 1102. In an exemplary embodiment, different mechanisms may be employed to pass the medical solution into cartridge 1102. For example, the medical solution may be passed into cartridge 1102 by pushing elastic membrane 1002 of FIG. 10. In an exemplary embodiment, cartridge 1102 may further be equipped with a valve or a button to enable (for example, by opening the valve or pushing the button) a flow of the medical solution into cartridge 1102. An exemplary flow of the medical solution into cartridge 1102 may be allowed until an amount of the medical solution inside cartridge 1102 may reach predetermined amount 1103. In an exemplary embodiment, graduated marks 1104 may be utilized to determine whether an amount of the medical solution has reached the predetermined amount. For example, a graduated mark 1106 may indicate that an amount of the medical solution inside cartridge 1102 has reached predetermined amount 1103.

An exemplary flow of the medical solution into cartridge 1102 may be stopped (for example, by releasing elastic membrane 1002 or an exemplary button of cartridge 1102, or closing an exemplary valve of cartridge 1102) when the amount of the medical solution inside cartridge 1102 reaches predetermined amount 1103. In an exemplary embodiment, cartridge 1102 may pass predetermined amount 1103 to microneedle 508 after the amount of the medical solution inside cartridge 1102 reaches predetermined amount 1103 to inject the medical solution into skin 110.

While the foregoing has described what are considered to be the best mode and/or other examples, it is understood that various modifications may be made therein and that the subject matter disclosed herein may be implemented in various forms and examples, and that the teachings may be applied in numerous applications, only some of which have been described herein. It is intended by the following claims to claim any and all applications, modifications, and variations that fall within the true scope of the present teachings.

Unless otherwise stated, all measurements, values, ratings, positions, magnitudes, sizes, and other specifications that are set forth in this specification, including in the claims that follow, are approximate, not exact. They are intended to have a reasonable range that is consistent with the functions to which they relate and with what is customary in the art to which they pertain.

The scope of protection is limited solely by the claims that now follow. That scope is intended and should be interpreted to be as broad as is consistent with the ordinary meaning of the language that is used in the claims when interpreted in light of this specification and the prosecution history that follows and to encompass all structural and functional equivalents.

Except as stated immediately above, nothing that has been stated or illustrated is intended or should be interpreted to cause a dedication of any component, step, feature, object, benefit, advantage, or equivalent to the public, regardless of whether it is or is not recited in the claims.

It will be understood that the terms and expressions used herein have the ordinary meaning as is accorded to such terms and expressions with respect to their corresponding respective areas of inquiry and study except where specific meanings have otherwise been set forth herein. Relational terms such as first and second and the like may be used solely to distinguish one entity or action from another without necessarily requiring or implying any actual such relationship or order between such entities or actions. The terms “comprises,” “comprising,” or any other variation thereof, are intended to cover a non-exclusive inclusion, such that a process, method, article, or apparatus that comprises a list of elements does not include only those elements but may include other elements not expressly listed or inherent to such process, method, article, or apparatus. An element proceeded by “a” or “an” does not, without further constraints, preclude the existence of additional identical elements in the process, method, article, or apparatus that comprises the element.

The Abstract of the Disclosure is provided to allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in various implementations. This is for purposes of streamlining the disclosure, and is not to be interpreted as reflecting an intention that the claimed implementations require more features than are expressly recited in each claim. Rather, as the following claims reflect, inventive subject matter lies in less than all features of a single disclosed implementation. Thus, the following claims are hereby incorporated into the Detailed Description, with each claim standing on its own as a separately claimed subject matter.

While various implementations have been described, the description is intended to be exemplary, rather than limiting and it will be apparent to those of ordinary skill in the art that many more implementations and implementations are possible that are within the scope of the implementations. Although many possible combinations of features are shown in the accompanying figures and discussed in this detailed description, many other combinations of the disclosed features are possible. Any feature of any implementation may be used in combination with or substituted for any other feature or element in any other implementation unless specifically restricted. Therefore, it will be understood that any of the features shown and/or discussed in the present disclosure may be implemented together in any suitable combination. Accordingly, the implementations are not to be restricted except in light of the attached claims and their equivalents. Also, various modifications and changes may be made within the scope of the attached claims.

What is claimed is:

1. A medical injection apparatus, comprising:
a first reservoir configured to contain a saline solution;
a second reservoir configured to contain medicine;
a static mixer configured to:
receive the saline solution from the first reservoir;
receive the medicine from the second reservoir; and
obtain a medical solution by mixing the medicine with the saline solution; and
an injection unit configured to:
receive the medical solution from the static mixer; and
inject the medical solution into a skin of a subject.

2. The medical injection apparatus of claim 1, wherein the first reservoir is coupled in parallel with the second reservoir.

3. The medical injection apparatus of claim 2, further comprising a valve connected between the first reservoir, the second reservoir, and the static mixer, the valve configured to:
seal each of the first reservoir, the second reservoir, and the static mixer with respect to one another responsive to the valve being closed; and
pass the saline solution from the first reservoir and pass the medicine from the second reservoir to the static mixer responsive to the valve being opened.

4. The medical injection apparatus of claim 3, wherein the medicine comprises a liquid medicine.

5. The medical injection apparatus of claim 4, wherein the liquid medicine comprises a solution of botulinum toxin.

6. The medical injection apparatus of claim 1, wherein:
the first reservoir is mounted on the second reservoir; and
the second reservoir is mounted on the static mixer.

7. The medical injection apparatus of claim 6, wherein:
the first reservoir comprises an elastic balloon; and
the medical injection apparatus further comprises one of a barrier or a semipermeable membrane placed between the elastic balloon and the second reservoir, each of the barrier and the semipermeable membrane configured to pass the saline solution from the elastic balloon to the second reservoir responsive to pressing the elastic balloon.

8. The medical injection apparatus of claim 6, further comprising a valve connected between the first reservoir and the second reservoir, the valve configured to:
seal the first reservoir with respect to the second reservoir responsive to the valve being closed; and
push the medicine from the second reservoir to the static mixer by passing the saline solution from the first reservoir to the second reservoir responsive to the valve being opened.

9. The medical injection apparatus of claim 8, wherein the medicine comprises a botulinum toxin powder.

10. The medical injection apparatus of claim 1, wherein the injection unit comprises:
one or more chambers;
a primary pipe connected to the static mixer;
one or more secondary pipes connecting the primary pipe to the one or more chambers, each respective secondary pipe of the one or more secondary pipes configured to:
receive the medical solution from the static mixer through the primary pipe; and
pass the medical solution to a respective chamber of the one or more chambers; and
a plurality of microneedle arrays attached to the one or more chambers, a tip of each respective microneedle in each respective microneedle array of the plurality of microneedle arrays extruding from a surface of each respective chamber of the one or more chambers.

11. The medical injection apparatus of claim 10, wherein each of the plurality of microneedle arrays comprises an array of hollow microneedles.

12. The medical injection apparatus of claim 10, further comprising:
a valve connected between the static mixer and the primary pipe; and
a compressed spring mounted on the first reservoir, the compressed spring configured to push the medical solution from the static mixer into the primary pipe responsive to the valve being opened.

13. The medical injection apparatus of claim 10, further comprising one or more elastic membranes, each respective elastic membrane of the one or more elastic membranes mounted on a respective chamber of the one or more chambers and configured to push the medical solution from the respective chamber into each microneedle in a respective microneedle array of the plurality of microneedle arrays responsive to the respective elastic membrane being pushed.

14. The medical injection apparatus of claim 10, further comprising a plurality of cartridges, each respective cartridge of the plurality of cartridges connected between a respective chamber of the one or more chambers and a respective microneedle in a respective microneedle array of the plurality of microneedle arrays and configured to:
receive a predetermined amount of the medical solution from the respective chamber; and
pass the predetermined amount of the medical solution to the respective microneedle.

15. A method for medical injection, comprising:
containing a saline solution in a first reservoir;
containing medicine in a second reservoir;
receiving the saline solution from the first reservoir at a static mixer;
receiving the medicine from the second reservoir at the static mixer;
obtaining, utilizing the static mixer, a medical solution by mixing the medicine with the saline solution;
receiving the medical solution from the static mixer at an injection unit; and
injecting, utilizing the injection unit, the medical solution into a skin of a subject.

16. The method of claim 14, further comprising:
mounting the first reservoir on the second reservoir; and
mounting the second reservoir on the static mixer.

17. The method of claim 15, wherein receiving the saline solution from the first reservoir at the static mixer and receiving the medicine from the second reservoir at the static mixer comprise:
pushing the medicine from the second reservoir to the static mixer, comprising:
passing the saline solution from the first reservoir to the second reservoir by opening a first valve connected between the first reservoir and the second reservoir.

18. The method of claim 16, wherein receiving the medical solution from the static mixer at the injection unit comprises:
passing the medical solution from the static mixer to a primary pipe connected to the static mixer;
passing the medical solution from the primary pipe to a respective chamber of one or more chambers through a respective pipe of one or more pipes connecting the primary pipe to the one or more chambers; and
passing the medical solution from the respective chamber to each respective hollow microneedle in a respective hollow microneedle array of a plurality of hollow microneedle arrays attached to the one or more chambers, a tip of the each respective microneedle extruding from a surface of the respective chamber.

19. The method of claim 17, wherein injecting the medical solution comprises:
mounting a compressed spring on the first reservoir;
connecting a second valve between the static mixer and the primary pipe; and
pushing, utilizing the compressed spring, the medical solution from the static mixer into and the primary pipe by opening the second valve.

20. The method of claim 17, wherein injecting the medical solution comprises:
mounting each respective elastic membrane of one or more elastic membranes on a respective chamber of the one or more chambers; and
pushing the medical solution from the respective chamber into each microneedle in a respective microneedle array of the plurality of microneedle arrays by pushing a respective elastic membrane of the one or more elastic membranes.
